(19) United States
(12) Patent Application Publication
(10) Pub. No.: US 2024/0062900 A1
(43) Pub. Date: Feb. 22, 2024

(54) INFORMATION PROCESSING DEVICE, INFORMATION PROCESSING METHOD, AND COMPUTER PROGRAM PRODUCT

(71) Applicant: KABUSHIKI KAISHA TOSHIBA, Tokyo (JP)

(72) Inventors: Masaaki TAKADA, Kashiwa Chiba (JP); Miho SAKO, Kawasaki Kanagawa (JP); Hisashi YAMADA, Kawasaki Kanagawa (JP)

(73) Assignee: KABUSHIKI KAISHA TOSHIBA, Tokyo (JP)

(21) Appl. No.: 18/173,825

(22) Filed: Feb. 24, 2023

(30) Foreign Application Priority Data
Aug. 18, 2022 (JP) 2022-130453

(51) Int. Cl. G16H 50/20; G16H 70/60

(52) U.S. Cl. CPC G16H 50/20; G16H 70/60

(57) ABSTRACT

An information processing device according to an embodiment includes a hardware processor coupled to a memory. The hardware processor estimates morbidity representing a probability of a subject being suffering from a specific disease. The morbidity is estimated on the basis of: a first probability model representing a relation between a first physical quantity associated with the specific disease and a second physical quantity to be measured, a second probability model representing a relation between the first physical quantity and information about whether the subject is suffering from the specific disease, a prior probability of morbidity representing a probability of the subject being suffering from the specific disease in a situation where no information has been obtained with respect to the first physical quantity or the second physical quantity related to the subject, and the second physical quantity obtained by measuring the subject.

### CROSS-REFERENCE TO RELATED APPLICATIONS

This application is based upon and claims the benefit of priority from Japanese Patent Application No. 2022-130453, filed on Aug. 18, 2022; the entire contents of which are incorporated herein by reference.

### FIELD

Embodiments described herein relate generally to an information processing device, an information processing method, and a computer program product.

### BACKGROUND

Technologies for outputting information for diagnosing a specific disease (illness) by using biomarkers have been developed. Biomarkers are biological substances such as proteins or genes, and are used as indicators of the presence of disease, change in symptoms, and the effectiveness of treatment.

For example, in a situation where a physical quantity related to the biomarker cannot be measured directly, a measurable physical quantity that changes according to the biomarker is measured.

By using a graph representing the relation between this physical quantity and a physical quantity related to the biomarker, the physical quantity related to the biomarker is estimated from the measured physical quantity.

A technology that applies a Bayesian model to estimate this graph or estimate the physical quantity related to the biomarker by using the graph has been developed.

### BRIEF DESCRIPTION OF THE DRAWINGS

FIG. 1 is a diagram illustrating an example of a calibration curve used in a comparative example;

FIG. 2 is a block diagram of an information processing device according to an embodiment;

FIG. 3 is a flowchart of a model estimation process in the embodiment;

FIG. 4 is a flowchart of a probability estimation process in the embodiment; and

FIG. 5 is a hardware configuration diagram of the information processing device according to the embodiment.

### DETAILED DESCRIPTION

An information processing device according to an embodiment includes one or more hardware processors. The one or more hardware processors are configured to estimate morbidity representing a probability of a subject being suffering from a specific disease. The morbidity is estimated on the basis of: a first probability model representing a relation between a first physical quantity associated with the specific disease and a second physical quantity to be measured, a second probability model representing a relation between the first physical quantity and information about whether the subject is suffering from the specific disease, a prior probability of morbidity representing a probability of the subject being suffering from the specific disease in a situation where no information has been obtained with respect to the first physical quantity or the second physical quantity related to the subject, and the second physical quantity obtained by measuring the subject.

Hereinafter, a preferred embodiment of an information processing device according to the invention will be described in detail with reference to the accompanying drawings.

As described above, biomarkers are used as indicators of the presence of disease, change in symptoms, and the effectiveness of treatment. A physical quantity representing the amount of biomarker corresponds to the physical quantity associated with a specific disease (first physical quantity). For example, the physical quantity representing the amount of biomarker is the concentration of biomarker in the body fluid. However, it is not limited thereto, and the physical quantity representing the amount of biomarker may be any physical quantity. In the following example, concentration is mainly used.

For example, a tumor marker is a biomarker related to cancer. Proteins characteristically produced by cancer cells, cells that react with cancer cells, or the like are used as tumor markers. The amount (concentration) of tumor marker is measured to obtain information on the presence of cancer and the progression of cancer.

Therefore, in the tumor marker test, the concentration of tumor markers in the body fluids such as blood and urine is measured, to obtain information related to the detection and progression of cancer. The disease is diagnosed by a combination of the information described above, and the results obtained from the other medical examinations, image inspection, and the like.

Various substances in the body such as genes, enzymes, hormones, deoxyribonucleic acid (DNA), messenger Ribonucleic acid (mRNA), micro Ribonucleic acid (miRNA), and long noncoding Ribonucleic acid (lncRNA) have been studied and developed as biomarkers for cancer in addition to proteins.

miRNA is a single-stranded nucleic acid of about 17 to 25 bases, and functions to regulate gene expression. Moreover, it has been reported that the type and expression level of miRNAs change from the early stages of cancer. Thus, a technology using miRNAs as biomarkers has attracted much attention as a technology that enables ultra-early detection of cancer.

Moreover, biomarkers have been studied and developed for various diseases such as Alzheimer's disease, heart disease, and stroke, in addition to cancer.

For diagnosing disease by using biomarkers, it is necessary to accurately measure the concentration of a substance in the body, which is specifically related to the disease. A measurable physical quantity (second physical quantity) that varies with concentration may be used to measure the concentration of a substance in the body. In the following, such a physical quantity may be referred to as a measured physical quantity. A graph representing the relation between the measured physical quantity and concentration is referred to as a calibration curve or a standard curve.

Concentration is estimated by, for example, the following procedure. When concentration can be estimated by the following procedure, the concentration is the physical quantity representing the amount of biomarker.
A measured physical quantity is measured by using a standard sample with a known concentration, and a calibration curve is obtained.
A measured physical quantity of a sample with an unknown concentration is measured, and the concentration corresponding to the measured physical quantity is obtained by using the calibration curve.

The measured physical quantity includes, for example, intensity of light, voltage, current, and time associated with reaction. The time associated with reaction is time associated with reaction (reaction time) using a sample taken from a subject.

In a case where the concentration is very small, the substance is amplified on the basis of the Polymerase Chain Reaction (PCR) method, Loop-Mediated Isothermal Amplification (LAMP) method, and the like. This amplification process is a target to be measured.

As a measurement method in a case where miRNAs are used as biomarkers, a technology has been developed, in which nucleic acid is amplified on the basis of the LAMP method, the rise time of the electrical signal (an example of reaction time) is measured, and the miRNA concentration is estimated from the relation (calibration curve) between the rise time and concentration.

In the estimation of concentration using a calibration curve, the concentration may not be obtained accurately due to various reasons as illustrated in the following (R1) to (R3).
(R1) If noise is added to the measured physical quantity itself, and if noise is added during the process of measuring the measured physical quantity, an error occurs in the measured physical quantity that has been measured. For example, the amplification of nucleic acid by the LAMP method can be affected by various measurement conditions such as the reagent lot, reagent storage conditions, temperature, and centrifugation conditions, in addition to the concentration of the target substance. Moreover, to measure the rise time of the electrical signal, the electrical signal must be measured at regular time intervals (for example, one minute intervals). Hence, only discrete values can be calculated.
(R2) The error increases with a decrease in the number of times the measured physical quantity used for estimating the calibration curve and the measured physical quantity used for measuring the subject are measured. The accuracy of the calibration curve increases with an increase in the number of times the sample with a known concentration used for estimating the calibration curve is measured. On the other hand, the calibration curve may change day to day. Hence, the calibration curve needs to be re-estimated regularly, and the measurement requires time and high cost. Therefore, for example, a small number of samples such as three samples may be used, and in such a case, the error is increased. Similarly, the subject can also be measured accurately with an increase in the number of times the subject is measured. However, due to time and cost constraints, it is difficult to measure the subject many times. When the number of measurements is small, the error may be increased.
(R3) The calibration curve is estimated by fitting the calibration curve to a model such as a simple regression model and a logistic regression model. The model includes assumptions such as linearity and homoscedasticity, and the assumptions may deviate from reality. In such a case, even if the calibration curve is estimated by finding the conditions that make it possible to reduce variations, or by increasing the number of measurements, an error can still occur in the concentration estimated by the estimated calibration curve.

In a case where the error in the estimated concentration is large due to such various reasons described above, it is difficult to accurately estimate the presence of disease or the progression of disease on the basis of the calculated concentration value.

In the present embodiment, even if the error in the estimated concentration is large, it is possible to accurately estimate the presence of disease or the progression of disease. In the present embodiment, the presence of disease or the progression of disease is robustly estimated by using a Bayesian model.

In the present embodiment, the following four inputs (I1) to (I4) are used to estimate the morbidity that represents the probability of being suffering from a specific disease. Then, the morbidity is calculated by calculating the product and the sum (or integral) of multiple probabilities described below.
(I1) A probability model MA (first probability model) that associates the measured physical quantity with concentration
(I2) A probability model MB (second probability model) that associates concentration with the presence of disease
(I3) Morbidity in a situation where no information has been obtained with respect to the measured physical quantity or concentration of the subject (prior probability of morbidity)
(I4) Measured physical quantity of the subject

There may be also a method of diagnosing disease after obtaining a point estimate of the concentration as a representative value such as an average value or a median value of the concentration distribution by using the probability model MA to estimate the calibration curve or to estimate the concentration of biomarker. In a situation where the measurement error (noise) is small, such a method may not pose a problem. On the other hand, for example, in a situation where the measurement error is large, the noise in the estimated concentration may affect diagnosis of disease.

In contrast, in the present embodiment, multiple probability models (probability model MA and probability model MB) can be integrated to estimate the morbidity. In other words, in the present embodiment, the morbidity is estimated directly, without performing point estimation on the concentration (for example, estimation of concentration by the probability model MA alone). Consequently, it is possible to suppress the influence of noise, and more accurately estimate information for diagnosing disease.

A disease as a target of estimating the morbidity may be any disease. For example, a disease as a target of estimating the morbidity is cancer. Cancer includes, for example, one or more of breast cancer, pancreatic cancer, lung cancer, stomach cancer, colon cancer, prostate cancer, ovarian cancer, esophageal cancer, liver cancer, biliary tract cancer, bladder cancer, brain tumor, and sarcoma. Early detection of cancer leads to improved survival rates, so that the development of biomarkers for early detection is eagerly awaited.

In the following, an example of diagnosing cancer by measuring the electrochemical reaction time and estimating the miRNA concentration will be mainly described. In this example, the measured physical quantity is reaction time, the biomarker is a specific miRNA concentration, and the specific disease is cancer.

First, an example (comparative example) of a method of estimating the presence of disease by using a calibration curve without using a Bayesian model will be described. In the comparative example, the following procedure is used to diagnose cancer.
(C1) Two or more standard samples with a known concentration are prepared, and the reaction time of each of the standard samples is measured once or more.
(C2) The calibration curve is estimated, by a simple regression model, on the basis of data on the measured concentration and reaction time.
(C3) The reaction time of a sample with an unknown concentration is estimated once or more.
(C4) The concentration corresponding to the reaction time measured at (C3) is estimated by using the calibration curve.
(C5) The estimated concentration is compared with the preset threshold value, and the presence of cancer is estimated by using a result of the comparison.

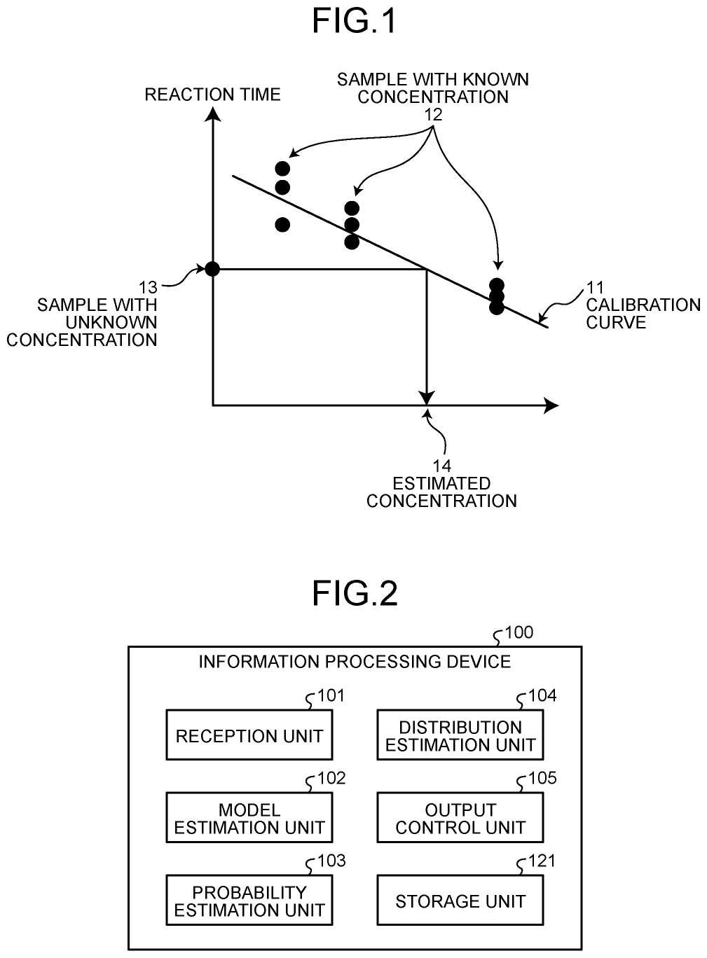

FIG. 1 is a diagram illustrating an example of a calibration curve used in the comparative example. A calibration curve 11 in FIG. 1 illustrates the relation between concentration and reaction time. The calibration curve 11 is estimated from a plurality of samples 12 with a known concentration. By using the estimated calibration curve 11, the concentration (estimated concentration 14) corresponding to the reaction time of a sample 13 with an unknown concentration is estimated.

The process in the comparative example will be further described by using mathematical expressions.

The reaction time is represented by Y ∈ R, the miRNA concentration is represented by X ∈ R, and the presence of cancer is represented by S={healthy individual, patient}. The materialized values (actually measured values) of the reaction time and miRNA concentration are expressed in lower case letters such as y and x. Preprocessing such as taking a logarithm and normalization may be performed on the reaction time and miRNA concentration in advance.

At (C1), a set of data {(xi, yi)} (1≤i≤n) of the value of miRNA concentration of a standard sample with a known concentration and the measured reaction time is obtained.

At (C2), by using the obtained data, the simple regression model illustrated in the following equation (1) is estimated. μ\hat{ }₀ and μ\hat{ }₁ are calculated by the following equation (2).

Y=\hat{β}₀+\hat{β}₁X (1)

At (C3), the reaction time y of a sample with an unknown concentration is measured. When the reaction time is measured multiple times, the average is taken as y.

At (C4), by using a simple regression model, concentration x\hat{ } is estimated by the following equation (3).

$$\hat{x} = \frac{y - {\hat{β}}_{0}}{{\hat{β}}_{1}} \qquad ( 3 )$$

At (C5), the estimated concentration x\hat{ } is compared with the preset threshold value xₜₕ. For example, when the morbidity of cancer increases with an increase in the concentration, the person is considered as a healthy individual if it is x\hat{ }<xₜₕ, and the person is considered as a cancer patient if it is x\hat{ }≥xₜₕ. When the morbidity of cancer increases with a decrease in the concentration, the opposite is true. The equal sign may be in either equation.

The calibration curve is not limited to the simple regression model, and another model such as a logistic regression model may also be used.

As described above, in the method of the comparative example, it may not be possible to accurately estimate the presence cancer, if at least one of the reaction time and calibration curve is inaccurate. In the present embodiment, it is possible to more accurately (robustly) estimate the presence of cancer, even if at least one of the reaction time and calibration curve is inaccurate.

Hereinafter, details of the present embodiment will be described. FIG. 2 is a block diagram illustrating an example of a structure of an information processing device 100 according to the present embodiment. As illustrated in FIG. 2, the information processing device 100 includes a reception unit 101, a model estimation unit 102, a probability estimation unit 103, a distribution estimation unit 104, an output control unit 105, and a storage unit 121.

The reception unit 101 receives input of various types of data used in the information processing device 100. For example, the reception unit 101 receives data on a sample with a known concentration (a set of data of concentration and reaction time) and data on a sample with an unknown concentration (measured reaction time).

The model estimation unit 102 estimates the probability model used to estimate the morbidity. For example, the model estimation unit 102 estimates the probability model MA by using miRNA concentration obtained in advance (such as miRNA concentration of a standard sample with a known concentration) and reaction time obtained in advance (such as reaction time obtained by measuring a standard sample with a known concentration).

Moreover, the model estimation unit 102 estimates the probability model MB by using the measured physical quantity (reaction time) obtained by measuring each of a healthy individual who is not suffering from a specific disease and a patient who is suffering from the specific disease.

The probability estimation unit 103 estimates the morbidity of the subject by using Bayes' theorem, on the basis of the probability model MA, the probability model MB, the prior probability of morbidity, and the measured physical quantity (reaction time) obtained by measuring the subject. A method other than Bayes' theorem may also be used, as long as the method estimates the morbidity of the subject on the basis of the probability model MA, the probability model MB, the prior probability of morbidity, and the measured physical quantity (reaction time) obtained by measuring the subject.

The distribution estimation unit 104 estimates the distribution of miRNA concentration of the subject by using the probability model MA and the measured physical quantity (reaction time) obtained by measuring the subject. When the concentration distribution is not to be estimated or not to be output, the distribution estimation unit 104 need not be provided.

The output control unit 105 controls the output of various types of data used in the information processing device 100. For example, the output control unit 105 outputs the morbidity estimated by the probability estimation unit 103 and the distribution estimated by the distribution estimation unit 104. The output method by the output control unit 105 may be any method. For example, a method of displaying on a display device such as a liquid crystal display, a method of outputting to a recording medium by using an image forming device such as a printer, a method of transmitting data to an external device (such as a server, and other information processing device), and the like may be applied.

For example, each of the units described above (reception unit 101, model estimation unit 102, probability estimation unit 103, distribution estimation unit 104, and output control unit 105) is implemented by one or more hardware processors. For example, each of the units described above may also be implemented by causing a hardware processor such as a Central Processing Unit (CPU) to execute a computer program, namely, implemented by software. Each of the units described above may also be implemented by a processor such as a dedicated Integrated Circuit (IC), namely, implemented by hardware. Each of the units described above may also be implemented by a combination of software and hardware. In the case of using a plurality of processors, each of the processors may implement one of the units or two or more of the units.

The storage unit 121 stores various types of data used by the information processing device 100. For example, the storage unit 121 stores the data received by the reception unit 101, the data related to the probability model estimated by the model estimation unit 102, and the estimation results by the probability estimation unit 103 or distribution estimation unit 104.

The storage unit 121 may include any commonly used storage media such as a flash memory, a memory card, a Random Access Memory (RAM), a Hard Disk Drive (HDD), and an optical disc.

The configuration illustrated in FIG. 2 is merely an example, and is not limited thereto. For example, the units illustrated in FIG. 2 may be configured so that the units are provided in a dispersed manner. For example, the units may be provided on devices such that the structure used to estimate the probability model (such as the model estimation unit 102) and the structure used to estimate the morbidity using the estimated probability model (probability estimation unit 103, distribution estimation unit 104, and the like) are different from each other.

The details of the functions of the units described above will be further described. Hereinafter, details of the inputs (I1) to (I4) described above, and the details of the method of calculating morbidity from the inputs will be described.

As to Input (I1):

The probability model MA that represents the relation between reaction time and miRNA concentration is obtained by modeling the probability distribution P(Y| X=x) of the reaction time when the miRNA concentration is x. For example, when the reaction time follows a normal distribution, the average value of the normal distribution is proportional to the reaction time, and the standard deviation is constant (independent of concentration), P(Y| X=x) is expressed as follows. In this example, N(μ, σ) represents the normal distribution of an average p and a standard deviation a.

P(Y|X=x)=N(μ(x),σ),μ(x)=β₀+β₁x

Each parameter in the probability model MA is estimated by fitting the probability model MA to data obtained in advance. For example, the model estimation unit 102 estimates parameters β₀, β₁, and a so as the probability model MA fits the data, by using a set of data {(xi, yi)} (1≤i≤n) of the value of miRNA concentration of a standard sample with a known concentration and the measured reaction time.

The method of estimating the model that fits to data may be any method. For example, the model estimation unit 102 estimates the probability model MA by using one of a least squares method, a maximum likelihood method, a maximum a posteriori (MAP) estimation method, a Markov Chain Monte Carlo (MCMC) method, a variational Bayesian method, and an Expectation-Maximization (EM) algorithm.

A prior distribution may be required for the MAP estimation method, MCMC method, variational Bayesian method, and the like. As the prior distribution, one of no-information prior distribution, weak information prior distribution, and information prior distribution may be used. Moreover, a method of sequentially updating the prior distribution by using the previously estimated calibration curve, or a method of updating the prior distribution by using the average calibration curve in the past may also be used.

In a case where the standard deviation of the normal distribution depends on concentration, P(Y| X=x) may be modeled by using the standard deviation as a function σ(x) of the concentration as follows.

P(Y|X=x)=N(μ(x),σ(x)),μ(x)=β₀+β₁x

When, for example, the standard deviation is proportional to concentration, it may be σ(x)=α₀+α₁x. Because the standard deviation is greater than or equal to zero, σ(x) may be modeled by any of the following equations. α₀, α₁, α₂, and α₃ are parameters that define the model.

σ(x)=exp(α₀+α₁x)

σ(x)=α₂+exp(α₀+α₁x)

σ(x)=α₃+α₂/(1+exp(α₁(x−α₀)))

The probability model MA is not limited to the model that follows a normal distribution. For example, if there is a deviation in the distribution of reaction time, the model estimation unit 102 may model the probability model MA by using a skew normal distribution. The skew normal distribution is expressed by a position parameter μ(x), a scale parameter σ(x), and a shape parameter s(x), as follows.

P(Y|X=x)=SN(μ(x),σ(x),s(x))

If s(x)=0, the skew normal distribution is SN(μ(x), σ(x), 0), and coincides with the normal distribution N(μ(x), σ(x)). That is, the skew normal distribution is a generalized form of the normal distribution.

The shape parameter s(x) may also be modeled in any form. For example, the shape parameter s(x) may be modeled as s(x)=γ₀ as being independent of the concentration. The shape parameter s(x) may also be modeled as s(x)=γ₀+γ₁x as being linear to the concentration. γ₀ and γ₁ are parameters that define the model.

The model estimation unit 102 determines the shape of the probability model MA as described above, and estimates the parameters by using the MCMC method and the like on the basis of data on a sample with a known concentration. Consequently, the probability distribution P(Y| X=x) of the reaction time when the miRNA concentration is x, is modeled as the probability model MA.

As to Input (12):

The probability model MB associates the presence of disease with the miRNA concentration. The probability model MB is P(X| S=patient) and P(X| S=healthy individual) that each represent the distribution of miRNA concentration (concentration distribution) of the patient and healthy individual.

When the concentration distribution is approximated by a normal distribution, the probability model MB is modeled by using μ^{c}, σ^{c}, μ^{h}, and μ^{h} as illustrated below.

P(X|S=patient)=N(μ^{c},σ^{c})

P(X|S=healthy individual)=N(μ^{h},σ^{h})

μ^{c} and σ^{c} represent the average and standard deviation of a patient. μ^{h} and σ^{h} represent the average and standard deviation of a healthy individual.

If accurate concentration data on each patient and healthy individual is obtained in advance, the model estimation unit 102 estimates each parameter by fitting the probability model MB to the data. For example, the model estimation unit 102 estimates the parameters μ^{h}, σ^{h}, μ^{c}, and σ^{c} so as the probability model MB fits to a set of data {(sᵢ, xᵢ)} (1≤i≤n) of the presence of disease and concentration obtained in advance.

The method of estimating the model that fits to data may be any method. For example, similar to the probability model MA, the model estimation unit 102 estimates the probability model MB by using one of the least squares method, the maximum likelihood method, the MAP estimation method, the MCMC method, the variational Bayesian method, and the EM algorithm.

If accurate concentration data on each patient and healthy individual is not obtained in advance but it is possible to measure the accurate reaction time of the subject with known presence of disease, the model estimation unit 102 may collectively estimate the parameters of the probability model MA and probability model MB.

For example, by utilizing the fact that the set of data {(sᵢ, yᵢ)} (1≤i≤n) of the presence of disease and reaction time follows the models in the following equation (4) and equation (5), the model estimation unit 102 can estimate the parameters in P(Y| X=x) that is the probability model MA, and P(X| S=patient) and P(X| S=healthy individual) that are each the probability model MB.

$$P ( Y \vert S = PATIENT ) = {\int }_{- ∞}^{∞} P ( Y \vert X = x ) P ( X = x \vert S = PATIENT ) dx \qquad ( 4 )$$

$$P ( Y \vert S = HEALTHY INDIVIDUAL ) = {\int }_{- ∞}^{∞} P ( Y \vert X = x ) P ( X = x \vert S = HEALTHY INDIVIDUAL ) dx \qquad ( 5 )$$

The concentration distribution is not limited to a normal distribution, but may also be a skew normal distribution, a mixed normal distribution, and the like.

The model estimation unit 102 determines the shape of the probability model MB as described above, and estimates the parameters by using the MCMC method or the like on the basis of the reaction time or the accurate concentration of a sample with known presence of disease. Consequently, the distributions P(X| S=patient) and P(X| S=healthy individual) of the miRNA concentration of the patient and healthy individual are each modeled as the probability model MB.

As to Input (I3):

As described above, the prior probability of morbidity is the morbidity in a situation where no information has been obtained with respect to the measured physical quantity of the subject. For example, the prior probability of morbidity is estimated by using statistical information obtained in advance. For example, statistical information is information indicating the proportion of a plurality of subjects actually diagnosed with cancer among those who had a cancer screening in the past. The statistical information may be information obtained for each inspection agency (such as hospital) that performs screening, or may be information obtained for each broader area (region) such as municipality, prefecture, Japan, and the world.

The prior probability of morbidity may be calculated for each attribute information of the subject. For example, the attribute information is age and gender. In this case, the prior probability of morbidity corresponding to the attribute information of the subject is used to calculate the morbidity.

A fixed value (for example, 50%) may be used as the prior probability of morbidity, assuming that the morbidity is completely unknown.

As to Input (I4):

For the subject with an unknown miRNA concentration, the measured reaction time is used as the measured physical quantity. The reaction time may also be the result obtained by measuring the same subject multiple times (for example, the average value of the multiple measurement results).

As to Estimation of Morbidity

Next, details of the method of calculating morbidity from the inputs (I1) to (I4) will be described. For example, each input is expressed as follows.

(I1)P(Y|X=x)

(I2)P(X|S=patient),P(X|S=healthy individual)

(I3)P(S=patient),P(S=healthy individual)

(I4)Y=y

When the reaction time of the input (I4) is y, the morbidity P(S=patient| Y=y) of the subject is expressed by the following equation (6).

$$P ( S = PATIENT \vert Y = y ) = \int P ( S = PATIENT \vert X = x ) P ( X = x \vert Y = y ) dx = \int \frac{P ( X = x \vert S = PATIENT ) P ( S = PATIENT )}{P ( X = x )} \frac{P ( Y = y \vert X = x ) P ( X = x )}{P ( Y = y )} dx = \int \frac{P ( Y = y \vert X = x ) P ( X = x \vert S = PATIENT ) P ( S = PATIENT )}{P ( Y = y )} d x \qquad ( 6 )$$

In the second to third lines in equation (6), Bayes' theorem is used for P(S=patient| X=x) and P(X=x| Y=y).

Moreover, P(Y=y) in the last line is expressed by the following equation (7).

P(Y=y)∫P(Y=y|X=x)P(X=x|S=PATIENT)P(S=PATIENT)dx+∫P(Y=y|X=x)P(X=x|S=HEALTHY INDIVIDUAL)P(S=HEALTHY INDIVIDUAL)dx (7)

By changing the equation as described above, the probability estimation unit 103 calculates the morbidity P(S=patient| Y=y) of the subject, by integrating a function that includes at least some products of P(Y=y| X=x), P(X| S=patient), P(X| S=healthy individual), P(S=patient), and P(S=healthy individual). In this manner, the morbidity is estimated by using the equation P(S=patient| Y=y) for calculating the morbidity that is converted by Bayes' theorem as represented by the probability model MA (P(Y| X=x)), the probability model MB (P(X| S=patient), P(X| S=healthy individual)), and the prior probability of morbidity (P(S=patient), P(S=healthy individual)); and the measured physical quantity y.

For example, similar to the usual calculation of Riemann integrals, the probability estimation unit 103 divides concentration x by width Δ, which is small enough within a realistic range from xₘᵢₙ to xₘₐₓ, and calculates the value within the integral sign (∫) for each x. Then, the probability estimation unit 103 multiplies the calculated result by Δ, and calculates the sum total of the results of multiplication in all divisions.

A calculation similar to the above also applies when the reaction time of one subject is measured multiple k times. That is, it is assumed that the input (I4) is Y₁=y₁, . . . , Yₖ=yₖ, and each input independently follows the calibration curve model P(Y| X=x). Then, the following relation is satisfied.

P(Y₁=y₁, . . . ,Yₖ=yₖ|X=x)=P(Y=y₁|X=x) . . . P(Y=yₖ|X=x)

P(Y₁=y₁, . . . ,Yₖ=yₖ)=P(Y=y₁) . . . P(Y=yₖ)

Therefore, the morbidity P(S=patient| Y₁=y₁, . . . , Yₖ=yₖ) is calculated by the following equation (8). In this manner, similar to the above, the probability estimation unit 103 can calculate the cancer morbidity of the subject.

$$P ( S = PATIENT \vert {Y}_{1} = {y}_{1} , … , {Y}_{k} = {y}_{k} ) = \int \frac{\begin{matrix}P ( Y = {y}_{1} \vert X = x ) … P ( Y = {y}_{k} \vert X = x ) \\ P ( X = x \vert S = PATIENT ) P ( S = PATIENT )\end{matrix}}{P ( Y = {y}_{1} ) … P ( Y = {y}_{k} )} dx \qquad ( 8 )$$

Moreover, to measure the calibration curve and reaction time of the subject, the reaction time may be measured simultaneously by a plurality of electrodes k on the chip by one-time measurement. In this case, the reaction time is not a scalar Y ∈ R, but a k-dimensional vector Y ∈ R^{k}. If each dimension is independent, similar to the above, modeling and estimation may be performed assuming that each electrode follows the same model P(Y| X=x).

If there is a correlation between electrodes, it may be appropriate to treat Y while keeping it as a k-dimensional vector. In this case also, similar to the above, the probability estimation unit 103 can calculate the morbidity by the following equation (9).

$$P ( S = PATIENT \vert Y = y ) = \int \frac{P ( Y = y \vert X = x ) P ( X = x \vert S = PATIENT ) P ( S = PATIENT )}{P ( Y = y )} dx \qquad ( 9 )$$

The model representing the relation between reaction time and concentration at the time can be expressed as follows, by using a multidimensional normal distribution N(μ, Σ) of a mean vector μ ∈ R^{k} and a covariance vector Σ ∈ R^{kxk}. I is a k-dimensional vector in which all elements are 1.

P(Y|X=x)=N(μ(x)I,Σ),μ(x)=β₀+β₁x

In this manner, the probability estimation unit 103 can directly calculate the morbidity of the subject from the four inputs (I1) to (I4).

The output control unit 105 outputs the calculated morbidity on a display screen or on a recording medium such as paper. By using one or more threshold values, the output control unit 105 may also output information that indicates the morbidity risk, by dividing the information into two levels of high and low, three levels of high, middle, and low, or into more (four or more) levels.

An example of outputting the morbidity has been mainly described. The output control unit 105 may also output the concentration distribution of the subject estimated by the distribution estimation unit 104.

By using the following relational expression, the distribution estimation unit 104 can estimate the concentration distribution of the biomarker of a subject serving as a target.

P(X=x|Y=y)=P(Y=y|X=x)×P(X=x)/P(Y=y)

In this example, P(Y=y| X=x) can be calculated from the inputs (I1) and (I4), P(X=x)=P(X| S=patient) P(S=patient)+P(X| S=healthy individual) P(S=healthy individual) can be calculated from the inputs (I2) and (I3), and P(Y=y) (equation (7)) can be calculated from the inputs (I1) to (I4).

By outputting the concentration distribution estimated in this manner, the approximate range of concentration can be indicated even if, for example, the accurate concentration of the biomarker is not known. The output control unit 105 may output a graph, or a statistical value such as an average value, a median value, a mode value, and credible interval of the concentration distribution.

Binary values such as S={healthy individual, patient} have been used to diagnose disease. However, values for diagnosing disease are not limited to binary values. For example, values for diagnosing disease may also be obtained by defining the disease into three or more states (levels) according to the progression level of disease, such as S={level 0, level 1, level 2} and the like. In this case also, by using the same procedure as that when binary values are used, it is possible to estimate the probability at each level. For example, equation (7) is expressed by the sum of integrals of the number of levels. The numerator of the last line of equation (6) is expressed by the probability according to the level being a target of calculation of probability.

Examples of Other Diseases

In the examples described above, the disease to be diagnosed is cancer. The disease to be diagnosed is not limited to cancer, and may be any disease. In the following example, Alzheimer's disease is diagnosed.

It is considered that the majority of dementia cases are Alzheimer's disease. The Alzheimer's disease is believed to develop after the preclinical stage of Alzheimer's disease (preclinical AD) and mild cognitive impairment (MCI). Hence, early diagnosis and early intervention are considered important.

As biomarkers of Alzheimer's disease, amyloid β42, phosphorylated tau, total tau, and the like in cerebrospinal fluid or blood have been studied and developed.

A physical quantity representing the amount of these substances can be estimated by using the measured physical quantity measured by an enzyme immunoassay (ELISA), mass spectrometry, fluorescent-bead-based luminescence, electrochemiluminescence, and so forth.

Also for Alzheimer's disease, by applying the present embodiment utilizing a Bayesian model, it is possible to robustly estimate the presence of disease and the progression of disease from a very small amount of substances.

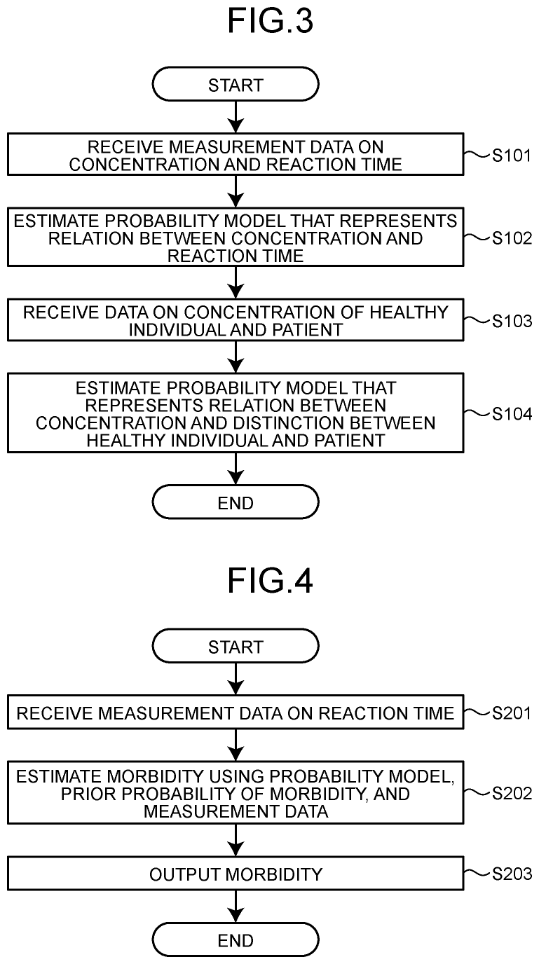

Next, a model estimation process performed by the information processing device 100 according to the present embodiment will be described. FIG. 3 is a flowchart illustrating an example of the model estimation process in the present embodiment.

The reception unit 101 receives an input of data on a set of concentration of a sample with a known concentration and reaction time (step S101). By using the input data, the model estimation unit 102 estimates the probability model MA that represents the relation between concentration and reaction time (step S102).

The reception unit 101 receives an input of data on concentration of each patient and healthy individual (step S103). By using the input data, the model estimation unit 102 estimates the probability model MB that represents relation between the concentration and the distinction between a healthy individual and a patient (step S104).

The estimation of the probability model MA (step S101 and step S102) and the estimation of the probability model MB (step S103 and step S104) need not be performed in the order illustrated in FIG. 3. The two probability models may also be estimated in reverse order or at independent timings.

The model estimation process may be performed at any time prior to the process performed by the probability estimation unit 103. The model estimation process may be performed periodically (for example, daily). The probability model MA may also be estimated every time the subject is measured, by using a device that can simultaneously measure the sample with a known concentration and the sample of the subject. The probability model MA may also be re-estimated, by estimating the probability model MA and the probability model MB by using a sample with known presence of disease as a preliminary verification, and estimating the probability model MA again when the subject with unknown presence of disease is measured. In this case, the former probability model MA is used to estimate the probability model MB, and the latter probability model MA is used to calculate the morbidity.

Next, a probability estimation process performed by the information processing device 100 according to the present embodiment will be described. FIG. 4 is a flowchart illustrating an example of the probability estimation process in the present embodiment.

The reception unit 101 receives data (measurement data) on the reaction time obtained by measuring the subject (step S201). The measurement data corresponds to the input (I4) described above. The probability estimation unit 103 estimates the morbidity by using the inputs (I1) to (I3), namely, the probability model MA, the probability model MB, the prior probability of morbidity, and the measurement data (step S202). The output control unit 105 outputs the estimated morbidity (step S203).

As described above, the present embodiment can more accurately estimate information for diagnosing disease.

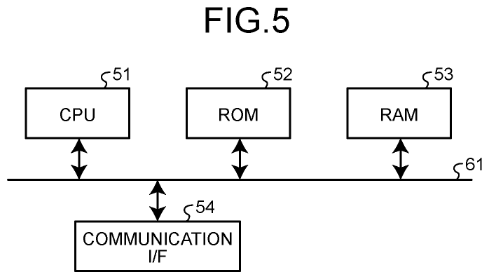

Next, a hardware configuration of the information processing device according to the embodiment will be described with reference to FIG. 5. FIG. 5 is an explanatory diagram illustrating an example of a hardware configuration of the information processing device according to the embodiment.

The information processing device according to the embodiment includes a control device such as a CPU 51, a storage device such as a Read Only Memory (ROM) 52 and a RAM 53, a communication interface (I/F) 54 that performs communication by connecting to a network, and a bus 61 that connects each unit.

The computer program executed by the information processing device according to the embodiment is provided by being incorporated in advance in the ROM 52 or the like.

The computer program executed by the information processing device according to the embodiment may also be recorded on a computer-readable recording medium such as a Compact Disk Read Only Memory (CD-ROM), a flexible disk (FD), a Compact Disk Recordable (CD-R), and a Digital Versatile Disk (DVD) in an installable or executable file format, and provided as a computer program product.

Moreover, the computer program to be executed by the information processing device according to the embodiment may be stored on a computer connected to a network such as the Internet, and provided by being downloaded through the network. Moreover, the computer program executed by the information processing device according to the embodiment may be provided or distributed via a network such as the Internet.

The computer program executed by the information processing device according to the embodiment can cause a computer to function as the units of the information processing device described above. In the computer, the CPU 51 can read a computer program from a computer-readable storage medium onto the main storage device and execute the computer program.

The configuration examples of the embodiment will be described below.

### (Configuration Example 1)

An information processing device includes a probability estimation unit that is configured to estimate morbidity representing a probability of a subject being suffering from a specific disease. The morbidity is estimated on the basis of: a first probability model representing a relation between a first physical quantity associated with the specific disease and a second physical quantity to be measured, a second probability model representing a relation between the first physical quantity and information about whether the subject is suffering from the specific disease, a prior probability of morbidity representing a probability of the subject being suffering from the specific disease in a situation where no information has been obtained with respect to the first physical quantity or the second physical quantity related to the subject, and the second physical quantity obtained by measuring the subject.

### (Configuration Example 2)

The information processing device according to the configuration example 1 further includes a distribution estimation unit that is configured to estimate distribution of the first physical quantity of the subject by using the first probability model and the second physical quantity obtained by measuring the subject.

### (Configuration Example 3)

The information processing device according to the configuration example 1 or 2 further includes a model estimation unit that is configured to estimate the first probability model by using the first physical quantity obtained in advance and the second physical quantity obtained in advance.

### (Configuration Example 4)

In the information processing device according to the configuration example 3, the model estimation unit is configured to perform the estimation of the first probability model by using one of a least squares method, a maximum likelihood method, a maximum a posteriori estimation method, a Markov Chain Monte Carlo method, a variational Bayesian method, and an Expectation-Maximization (EM) algorithm.

### (Configuration Example 5)

In the information processing device according to any one of the configuration examples 1 to 4, the first probability model is a model using a normal distribution or a skew normal distribution.

### (Configuration Example 6)

The information processing device according to any one of the configuration examples 1 to 5 further includes a model estimation unit that is configured to estimate the second probability model by using the second physical quantity obtained by measuring each of a healthy individual who is not suffering from the specific disease and a patient who is suffering from the specific disease.

### (Configuration Example 7)

In the information processing device according to the configuration example 6, the model estimation unit is configured to perform the estimation of the second probability model by using one of a least squares method, a maximum likelihood method, a maximum a posteriori estimation method, a Markov Chain Monte Carlo method, a variational Bayesian method, and an Expectation-Maximization (EM) algorithm.

### (Configuration Example 8)

In the information processing device according to any one of the configuration examples 1 to 7, the second probability model is a model using a normal distribution, a skew normal distribution, or a mixed normal distribution.

### (Configuration Example 9)

In the information processing device according to any one of the configuration examples 1 to 8, the prior probability of morbidity is estimated by using statistical information obtained in advance.

### (Configuration Example 10)

In the information processing device according to any one of the configuration examples 1 to 9, the second physical quantity includes intensity of light, voltage, current, and time, each being obtained by measuring a sample taken from the subject, the time being associated with reaction using the sample.

### (Configuration Example 11)

In the information processing device according to any one of the configuration examples 1 to 10, the probability estimation unit is configured to perform the estimation of the morbidity by integrating a function including a product of the first probability model, the second probability model, and the prior probability of morbidity.

### (Configuration Example 12)

In the information processing device according to any one of the configuration examples 1 to 11, the first physical quantity includes protein, gene, enzyme, hormone, deoxyribonucleic acid, messenger Ribonucleic acid (mRNA), micro RNA (miRNA), and long non-coding RNA (lncRNA) obtained from the subject.

### (Configuration Example 13)

In the information processing device according the configuration example 1, the specific disease includes one or more of breast cancer, pancreatic cancer, lung cancer, stomach cancer, colon cancer, prostate cancer, ovarian cancer, esophageal cancer, liver cancer, biliary tract cancer, bladder cancer, brain tumor, and sarcoma.

### (Configuration Example 14)

In the information processing device according the configuration example 1, the probability estimation unit is configured to perform the estimation of the morbidity by using Bayes' theorem on the basis of the first probability model, the second probability model, the prior probability of morbidity, and the second physical quantity obtained by measuring the subject.

### (Configuration Example 15)

In the information processing device according the configuration example 14, the probability estimation unit is configured to perform the estimation of the morbidity by using: an equation for calculating the morbidity converted by using Bayes' theorem such that the morbidity is represented by the first probability model, the second probability model, and the prior probability of morbidity; and the second physical quantity obtained by measuring the subject.

### (Configuration Example 16)

The information processing device according to the configuration example 1 further includes an output control unit that is configured to output the morbidity.

### (Configuration Example 17)

An information processing method implemented by a computer. The method includes estimating morbidity representing a probability of a subject being suffering from a specific disease. The estimating is performed on the basis of: a first probability model representing a relation between a first physical quantity associated with the specific disease and a second physical quantity to be measured, a second probability model representing a relation between the first physical quantity and information about whether the subject is suffering from the specific disease, a prior probability of morbidity representing a probability of the subject being suffering from the specific disease in a situation where no information has been obtained with respect to the first physical quantity or the second physical quantity related to the subject, and the second physical quantity obtained by measuring the subject.

### (Configuration Example 18)

A computer program that is executable by a computer. The computer program instructs the computer to estimate morbidity representing a probability of a subject being suffering from a specific disease. The morbidity is estimated on the basis of: a first probability model representing a relation between a first physical quantity associated with the specific disease and a second physical quantity to be measured, a second probability model representing a relation between the first physical quantity and information about whether the subject is suffering from the specific disease, a prior probability of morbidity representing a probability of the subject being suffering from the specific disease in a situation where no information has been obtained with respect to the first physical quantity or the second physical quantity related to the subject, and the second physical quantity obtained by measuring the subject.

While certain embodiments have been described, these embodiments have been presented by way of example only, and are not intended to limit the scope of the inventions. Indeed, the novel embodiments described herein may be embodied in a variety of other forms; moreover, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information processing device comprising:
one or more hardware processors configured to estimate morbidity representing a probability of a subject being suffering from a specific disease, the morbidity being estimated on the basis of
a first probability model representing a relation between a first physical quantity associated with the specific disease and a second physical quantity to be measured,
a second probability model representing a relation between the first physical quantity and information about whether the subject is suffering from the specific disease,
a prior probability of morbidity representing a probability of the subject being suffering from the specific disease in a situation where no information has been obtained with respect to the first physical quantity or the second physical quantity related to the subject, and
the second physical quantity obtained by measuring the subject.

2. The information processing device according to claim 1, wherein the one or more hardware processors are further configured to estimate distribution of the first physical quantity of the subject by using the first probability model and the second physical quantity obtained by measuring the subject.

3. The information processing device according to claim 1, wherein the one or more hardware processors are further configured to estimate the first probability model by using the first physical quantity obtained in advance and the second physical quantity obtained in advance.

4. The information processing device according to claim 3, wherein the one or more hardware processors are configured to perform the estimation of the first probability model by using one of a least squares method, a maximum likelihood method, a maximum a posteriori estimation method, a Markov Chain Monte Carlo method, a variational Bayesian method, and an Expectation-Maximization (EM) algorithm.

5. The information processing device according to claim 1, wherein the first probability model is a model using a normal distribution or a skew normal distribution.

6. The information processing device according to claim 1, wherein the one or more hardware processors are further configured to estimate the second probability model by using the second physical quantity obtained by measuring each of a healthy individual who is not suffering from the specific disease and a patient who is suffering from the specific disease.

7. The information processing device according to claim 6, wherein the one or more hardware processors are configured to perform the estimation of the second probability model by using one of a least squares method, a maximum likelihood method, a maximum a posteriori estimation method, a Markov Chain Monte Carlo method, a variational Bayesian method, and an Expectation-Maximization (EM) algorithm.

8. The information processing device according to claim 1, wherein the second probability model is a model using a normal distribution, a skew normal distribution, or a mixed normal distribution.

9. The information processing device according to claim 1, wherein the prior probability of morbidity is estimated by using statistical information obtained in advance.

10. The information processing device according to claim 1, wherein the second physical quantity includes intensity of light, voltage, current, and time, each being obtained by measuring a sample taken from the subject, the time being associated with reaction using the sample.

11. The information processing device according to claim 1, wherein the one or more hardware processors are configured to perform the estimation of the morbidity by integrating a function including a product of the first probability model, the second probability model, and the prior probability of morbidity.

12. The information processing device according to claim 1, wherein the first physical quantity includes protein, gene, enzyme, hormone, deoxyribonucleic acid, messenger Ribonucleic acid (mRNA), micro RNA (miRNA), and long non-coding RNA (lncRNA) obtained from the subject.

13. The information processing device according to claim 1, wherein the specific disease includes one or more of breast cancer, pancreatic cancer, lung cancer, stomach cancer, colon cancer, prostate cancer, ovarian cancer, esophageal cancer, liver cancer, biliary tract cancer, bladder cancer, brain tumor, and sarcoma.

14. The information processing device according to claim 1, wherein the one or more hardware processors are configured to perform the estimation of the morbidity by using Bayes' theorem on the basis of the first probability model, the second probability model, the prior probability of morbidity, and the second physical quantity obtained by measuring the subject.

15. The information processing device according to claim 14, wherein the one or more hardware processors are configured to perform the estimation of the morbidity by using
an equation for calculating the morbidity converted by using Bayes' theorem such that the morbidity is represented by the first probability model, the second probability model, and the prior probability of morbidity, and
the second physical quantity obtained by measuring the subject.

16. The information processing device according to claim 1, wherein the one or more hardware processors are further configured to output the morbidity.

17. An information processing method implemented by a computer, the method comprising:
estimating morbidity representing a probability of a subject being suffering from a specific disease, the estimating being performed on the basis of
a first probability model representing a relation between a first physical quantity associated with the specific disease and a second physical quantity to be measured,
a second probability model representing a relation between the first physical quantity and information about whether the subject is suffering from the specific disease,
a prior probability of morbidity representing a probability of the subject being suffering from the specific disease in a situation where no information has been obtained with respect to the first physical quantity or the second physical quantity related to the subject, and
the second physical quantity obtained by measuring the subject.

18. A computer program product comprising a non-transitory computer-readable recording medium on which a computer program executable by a computer is recorded, the computer program instructing the computer to:
estimate morbidity representing a probability of a subject being suffering from a specific disease, the morbidity being estimated on the basis of
a first probability model representing a relation between a first physical quantity associated with the specific disease and a second physical quantity to be measured,
a second probability model representing a relation between the first physical quantity and information about whether the subject is suffering from the specific disease,
a prior probability of morbidity representing a probability of the subject being suffering from the specific disease in a situation where no information has been obtained with respect to the first physical quantity or the second physical quantity related to the subject, and
the second physical quantity obtained by measuring the subject.
